(19) United States
(12) Patent Application Publication
(10) Pub. No.: US 2019/0134246 A1
(43) Pub. Date: May 9, 2019

(54) RECHARGEABLE, MICRO-DIFFUSER APPARATUS AND METHOD

(71) Applicant: Earl Vaughn Sevy, Cedar City, UT (US)

(72) Inventor: Earl Vaughn Sevy, Cedar City, UT (US)

(21) Appl. No.: 15/804,308

(22) Filed: Nov. 6, 2017

(51) Int. Cl. A61L 9/12

(52) U.S. Cl. CPC A61L 9/12

(57) ABSTRACT

A rechargeable, micro-diffuser apparatus and method provide a housing having a cage to hold an absorbent solid. The cage may include slots admitting air over absorbent beads. An aromatic liquid may drip or pour through an opening to the beads, which then absorb or adsorb that liquid. Ambient air evaporates or otherwise diffuses the aromatic liquid as aromatic vapor into surrounding air. The apparatus operates with no open container of bulk liquid, but only interstitially held liquid within polymeric molecules of absorbent beads or spread over an adsorbent surface.

### RELATED APPLICATIONS

This application incorporates by reference the following United States Patents and Patent Applications: U.S. Pat. No. 7,878,418, issued Feb. 1, 2011; U.S. Pat. No. D645,947, issued Sep. 27, 2011; U.S. Pat. No. 9,415,130, issued Aug. 16, 2016; application Ser. No. 15/373,035, filed Dec. 8, 2016; and application Ser. No. 14/850,789, filed Sep. 10, 2015.

### BACKGROUND

### Field of the Invention This invention relates to living space conditioning and, more particularly, to novel systems and methods for diffusing essential oils and other aromatic substances.

### Background Art

Automotive air fresheners represent small paper-felt objects treated with aromatic substances. Room fresheners may involve the same structures and chemical compositions. Often, rooms may be treated with wax candles containing aromatic materials. Paraffin containing aromatic oils has been used in both candle and electrical heating environments to circulate aromatic vapors. Aerosol sprays from propellant-pressurized cans have likewise been a staple of “room freshening.”

Direct diffusion of essential oils into living space has been documented in the references incorporated hereinabove by reference. Suffice it to say that fragrances, whether distributed by release from heated paraffinic materials, evaporated from paper-felt images dangling from rear view mirrors of automobiles, or distributed by aerosol spraying from pressurized cans in restrooms, are all attempts at conditioning living space.

Equipment size, weight, expense, and so forth may all be factors in selection of space conditioning by aromatic vapors. Oils may leak, spread, and react. They may ruin furniture, floors, handbags, upholstery, and so forth. Meanwhile, purses, glove boxes, dash boards, other mobile spaces, and the like do not serve as good locations for large diffusion equipment. Thus, it would be an advance in the art to create a comparatively small, portable, leak-proof or leak resistant, custom diffuser.

### BRIEF SUMMARY OF THE INVENTION

In view of the foregoing, in accordance with the invention as embodied and broadly described herein, a method and apparatus are disclosed in one embodiment of the present invention as including a housing shaped and sized to either fit onto or to act as the sealing lid for a container of an aromatic liquid, such as an essential oil. Such a system may include the housing as the largest component thereof, and have a closure or floor therebelow secured in any suitable manner by fasteners, glue, barbs, clips, interference fit, friction, screws, or the like to the housing.

The closure and housing together may contain a granular material such as absorbent beads (or even adsorbent) of a suitable material such as an acrylamide or other polymer that will effectively absorb, or even adsorb, a liquid selected to diffuse an aromatic vapor therefrom. Accordingly, the absorbent may absorb substantially all the available liquid making contact therewith, and diffuse it more slowly into the surrounding air.

The housing may include perforations that permit substantial passage of air through the housing and over the absorbent beads. Herein, absorbing is used in a chemical engineering sense. To that end, adsorbing is likewise. It is an alternate mechanism intended when the “absorbing” example is used.

The housing may act as the cap for a container such as a bottle. Nevertheless, the housing may be constructed to fit snuggly over a cap. In this latter embodiment, the housing may actually be used to turn the cap in order to free it from the container, and thereby open the container for applying an amount of the liquid contained therein to the absorbent beads in the housing. The container may be of any suitable size and shape, although the essential oils industry has apparently adopted a more-or-less standardized neck size, thread, and pour spout for containers of essential oils.

The housing may provide an interference fit, a frictional fit, a glued fit, a heated fit, or any other suitable arrangement to secure the housing to the cap. Together, they then move in rigid body motion. Rigid body motion is an engineering term of art and is used herein in that conventional engineering meaning.

A cap may conventionally include flutes or gripping elements (e.g., lands and grooves) on an outside surface thereof, which act like knurling (e.g., cross-cut rises and inclusions) to provide surface variation. This texturing provides grip or “purchase” on the cap in order to open it. Similarly, the housing, being somewhat larger than the cap may fit those lands and grooves or other texturing and may include similar flutes or grip elements on its outer surface.

In certain preferred embodiments, apertures may be slots between bars of a solid material forming the housing. The apertures or slots may be sized to be sufficiently small to resist or completely prevent any escape of absorbent beads therefrom within the housing. Such a cage still permits free passage of gases, such as ambient air therethrough.

In this way, ambient air may pick up molecules of aromatic liquid and carry them out as vapor. In one sense, one may think of the housing as forming a cage capturing absorbent beads, which in turn absorb aromatic liquids. Permitting passage through the housing by ambient air will carry the vapors of the aromatic liquid outside the housing and into a living space.

By living space is meant space occupied by humans or animals. For example, an automobile, an office, a purse, a room, or other space may be occupied by all or part of a human body. The result is exposure of human beings to prevailing aromas. An apparatus in accordance with the invention may be placed in any of these spaces, as small as a purse or briefcase or as large as a room or building in order to diffuse aromatic vapors therethroughout.

Size will necessarily effect the degree of influence exerted on a space by a diffuser in accordance with the invention. To that end, filling or charging apertures in a top of a housing may be formed as openings at the bottom of a depression. These permit a user to remove the cap and housing together from a container and pour from the container into the housing (through the charging apertures) a charge of oil. This charge is readily absorbed into the beads selected for that specific purpose.

The beads, by absorbing (or by adsorbing) the aromatic liquid (typically an essential oil), absorb the charge and draw it away from the housing and into the comparatively smaller porosity, oleophilic surface, or other surface-tension-dependent feature of the beads. Some liquids may actually chemically mix or even bond to elements of the molecules of the polymeric beads.

In some embodiments, the beads may hold the liquid by capillary action. In other mechanisms or embodiments, the liquid may be drawn into polymeric interstices or openings within larger polymeric molecules forming the beads. In this way, comparatively large amounts, even exceeding the mass of beads, may conceivably be absorbed thereby.

By whatever mode and to whatever degree selected and engineered, the aromatic liquid may be absorbed or otherwise retained. Another valuable utility is the ability of the absorbent beads to release molecules of the liquid by evaporation, by drying, or other physical and chemical operation of surrounding ambient air, the molecules that form the liquid and the aromatic vapor enter the surrounding environment.

The shape of the housing may include mechanisms to grip the cap, and selectively release therefrom in response to force, heat, or the like. Alternatively, the housing may actually be formed to be the cap, with the closure or floor of the housing actually serving to seal off by suitable treatment (e.g., washer, gasket, ring, ridge, disc, etc.) the container.

### BRIEF DESCRIPTION OF THE DRAWINGS

The foregoing features of the present invention will become more fully apparent from the following description and appended claims, taken in conjunction with the accompanying drawings. Understanding that these drawings depict only typical embodiments of the invention and are, therefore, not to be considered limiting of its scope, the invention will be described with additional specificity and detail through use of the accompanying drawings in which:

FIG. 1 is an exploded, perspective view of one embodiment of a system and apparatus in accordance with the invention;

FIG. 2 is a perspective view of an embodiment of a container and cap assembled, but exploded with respect to with a diffuser top housing, which is shown in an assembled view thereof;

FIG. 3 is an exploded, upper perspective view of the diffuser portion of an apparatus;

FIG. 4 is an exploded, lower perspective view thereof;

FIG. 5 is a top plan view thereof;

FIG. 6 is a bottom plan view thereof;

FIG. 7 is a side elevation view of the housing all sides being substantially identical, just rotated in phase;

FIG. 8 is a perspective view of an assembled system, with the container removed in order to load the system with an essential oil or other aromatic liquid from the container;

FIG. 9 is an exploded perspective view of optional shapes for a small, portable, diffuser system in accordance with the invention, absent the container or bottle supplying the aromatic liquid, showing various alternative embodiments of the housing;

FIG. 10 is an exploded, perspective view of one embodiment of a system having a collar and clip system for securing to a support a reservoir (bottle) by the neck, the bottle being capable of holding an aromatic liquid, essential oil, or the like, and receiving a rechargeable, micro diffuser attached as a cap thereon;

FIG. 11 is an exploded, perspective view of the system of FIG. 10, illustrating alternative embodiments of neck collars and clips fitting the neck of the container (bottle);

FIG. 12 is an exploded, perspective view of one embodiment of an assembled collar and clip prior to securement to a container; and

FIG. 13 is a perspective view thereof, assembled and installed on the container.

### DETAILED DESCRIPTION OF THE PREFERRED EMBODIMENTS

It will be readily understood that the components of the present invention, as generally described and illustrated in the drawings herein, could be arranged and designed in a wide variety of different configurations. Thus, the following more detailed description of the embodiments of the system and method of the present invention, as represented in the drawings, is not intended to limit the scope of the invention, as claimed, but is merely representative of various embodiments of the invention. The illustrated embodiments of the invention will be best understood by reference to the drawings, wherein like parts are designated by like numerals throughout.

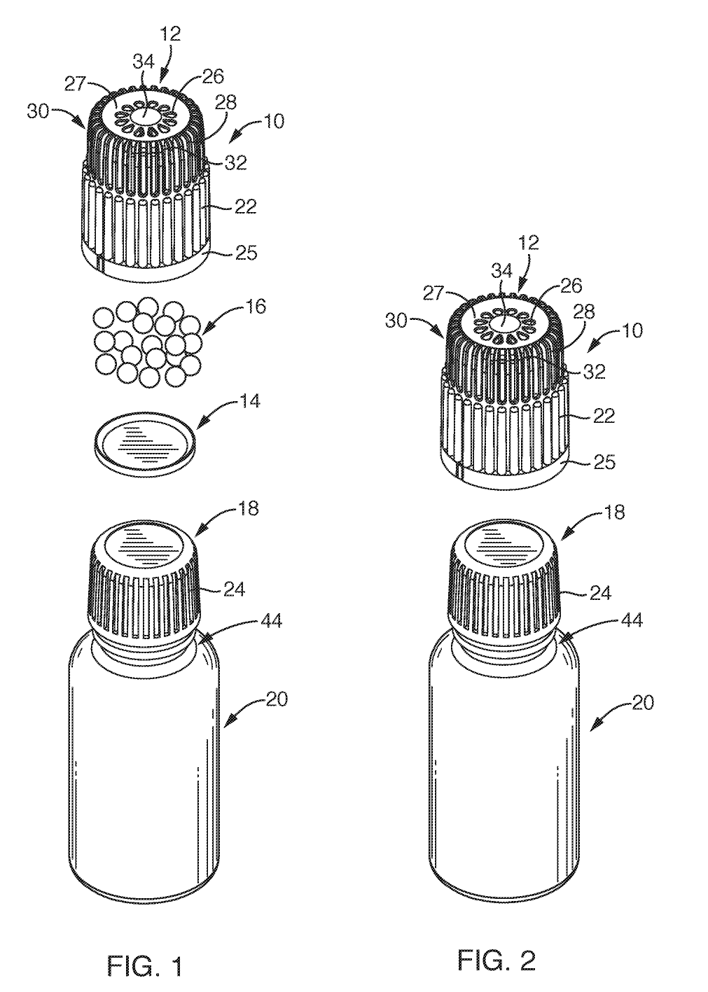

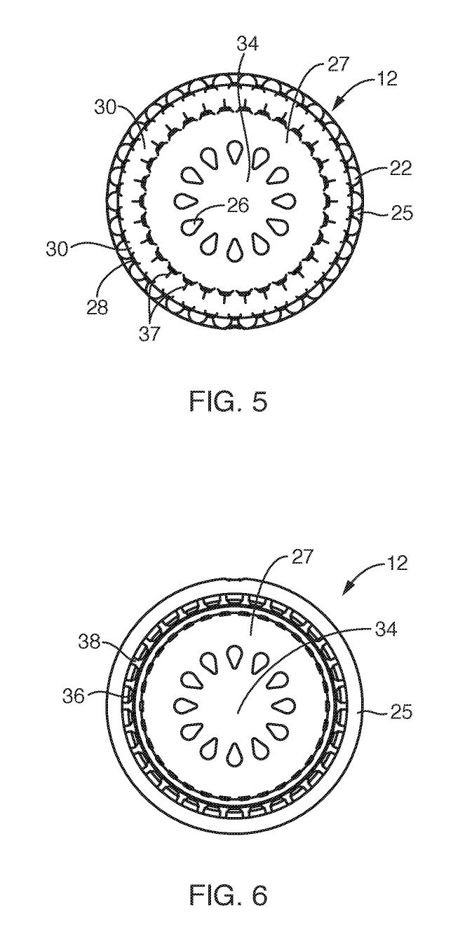

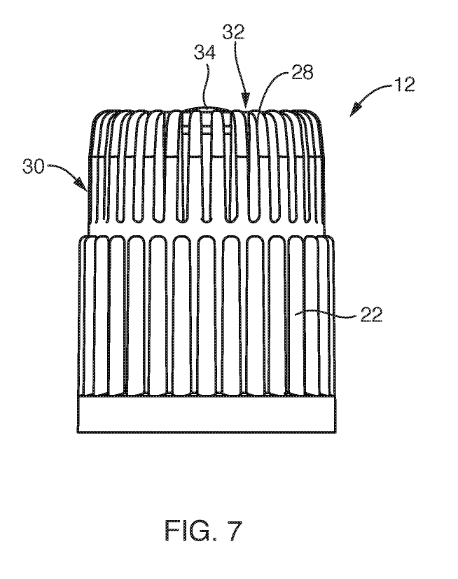

Referring to FIGS. 1 and 2, while referring generally to FIGS. 1 through 13, a system 10 in accordance with the invention may include a housing 12. The housing 12 may incorporate a floor 14 configured as a disc 14, plate 14, or other mechanism as a closure 14 of the housing 12.

One principal function of the closure 14 is to contain an absorbent 16 (or, alternatively, an absorbent) comprising particles 16, granules 16, or beads 16 of a material selected to attract and hold a liquid 40 of aromatic content 40 or essential oil 40. In the illustrated embodiment, the housing 12, once loaded with the absorbent 16 constituting granules 16, beads 16, or some other particulate absorbent 16, is closed by the floor 14. This floor 14 resists loss of the granules 16 out of the housing 12. Installation fixing the floor 14 may be done mechanically, such as by heating, gluing, snap fitting, barbs, screws, or the like.

In operation, the housing 12 fits in a firm, snug, and gripping manner over a cap 18 sealing a container 20. The container 20 is designed to contain a liquid 40 or content 40 that may be applied to the absorbent 16 to be absorbed thereby, and slowly released from the housing 12. All mention of an absorbent 16 as an example should be understood to include other carriers 16 and mechanisms, such as adsorbents 16 that rely on surface adherence, and the like.

In the illustrated embodiment, a grip 22 such as fluting 22 or knurling 22 on the outer surface of the housing 12 provides an ability to handle the housing 12 to press it onto the cap 18, remove it therefrom, and to use it as the element effectively rotating the cap 18 by action of the fingers of a user.

Likewise, a cap 18 will typically include a grip 24 such as fluting 24 formed integrally on the outside thereof in order to be able to secure and remove the cap 18 from the container 20. The housing 12 may be the cap 18 itself, or may be engineered to fit into the grip 24 or the fluting 24 of the cap 18 in order to fix the housing 12 with respect to the cap 18. Particularly in a circumferential direction this is required, but a connection also needs to be stable axially. The housing 12 may be left almost permanently secured to the cap 18, thus operating to open the cap 18 whenever desired. Alternatively, the housing 12 may be shaped with the threads on its interior surface matching the container. The housing 12 then replaces the lid 18 itself.

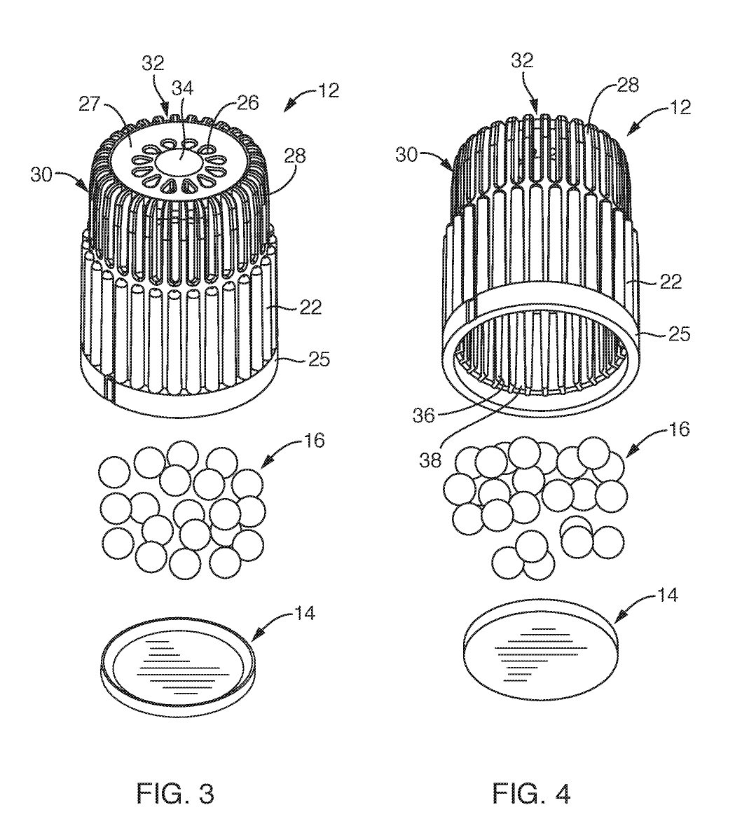

Referring to FIGS. 3 through 7, while continuing to refer generally to FIGS. 1 through 13, the housing 12 may be configured in any suitable arrangement. In the illustrated embodiment, a rim 25 circumscribes a lower edge of the housing 12. At an opposite (upper) end of the housing 12, apertures 26 are formed in a top 27 or top surface 27. The apertures 26 pass through the top 27, as the lowest points 26 or locations 26 in the top 27. Accordingly, any of the liquid 40 dripped or poured onto the top 27, will pass down through the apertures 26 promptly, being collected into and retained by the absorbent 16.

The vertical wall of the upper portion 30 or cage 30 of the housing 12 is constituted by alternating bars 28 defining apertures 32 or slots 32 between the bars 28. The cage 30 is designed to accomplish several functions, two of which include containment of the absorbent 16. In order to contain the absorbent 16, the cage 30 must have the bars 28 sufficiently close together. This is to assure that the beads 16, granules 16 or otherwise fashioned particles of the absorbent 16 cannot pass through the apertures 32 between the bars 28. A second function of the cage 30 is free passage of air through the apertures 32, in order to expose the beads 16 or absorbent 16 to ambient air. This assists in volatizing (evaporating) the aromatic liquid 40 contained in the absorbent 16.

To effect loading, filling, or otherwise treating the absorbent 16 with the liquid content 40 of the container 20, a dome 34 may be rounded, slotted, or otherwise shaped to urge the liquid 40 to pass downward through the apertures 26 in the top 27 to contact and be drawn to the absorbent 16 by surface tension. Inasmuch as the porosity of the absorbent 16 represents comparatively small openings therein, liquid 40 contacting the beads 16 will be drawn away from the apertures 26 to the absorbent 16.

One may see that the housing 12 may include a series of lands 36 or ridges 36 protruding inwardly therefrom. Between the lands 36 are grooves 38. The grooves 38 may be sized and shaped to fit the grip 24 or fluting 24 of the cap 18. In this way, an affirmative, mechanical connection is made between the lands 36 and the fluting 24. The fluting 24 will fit into the grooves 38, and directly oppose passage of lands 36 in any circumferential direction. Thus, the lands 36 push against the fluting 24, which will thereby twist the cap 18 for opening and closing on the container 20.

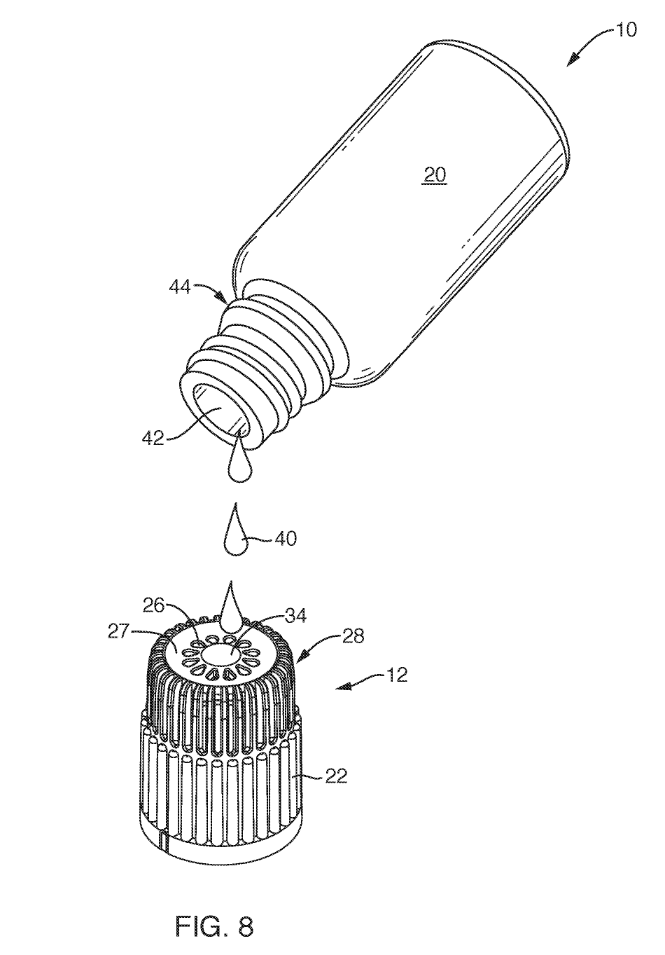

Referring to FIG. 8, while continuing to refer generally to FIGS. 1 through 13, the system 10 includes several steps in its operation. For example, in the illustrated embodiment, the housing 12 is fitted over the cap 18 to grip it. In fact, the housing 12 may be glued to the cap 18, but need not be. The housing 12 may be manufactured to provide an interference fit on the cap 18, thus providing a substantial frictional resistance to removal of the housing 12 from the cap 18. Nevertheless, with a certain degree of effort, the housing 12 may be separated from a frictional fit over the cap 18, in order to render the housing 12 reusable once the container 20 is empty of contents 40. Again, alternatively, the housing 12 may be threaded and sealed to directly replace the cap 18.

In the illustrated embodiment, drops 40 of the liquid 40 constituting the content 40 of the container 20 may be dripped or poured onto the top 27 of the housing 12. Typically, the top 27 is slanted inward and downward toward the apertures 26. The dome 34 slopes out and down to the apertures 26. That is, from the outside circumference of the top 27, the top 27 slopes downward toward the apertures 26. Likewise, the dome 34 may be highest at its center, and curve downward toward the apertures 26 as well.

In certain embodiments, narrow capillary slots may be formed with, as part of, or in place of the apertures 26, in order to draw liquids immediately downward. Nevertheless, it has been found completely effective to drop or drip drops 40 onto the top 27, where they tend to flow toward the apertures 26. Absorption into the beads 16 of the absorbent 16 captured within the cage portion 30 of the housing 12 is best if beads 16 fill the cage 30 up to the apertures 26.

To the extent that any residual oil or other content 40 may persist on the top 27, it may be wiped off with a finger and rubbed into hands as a harmless, and even beneficial, essential oil. Similarly, if a user desires, a tissue, cloth, or the like may be used to buff or dry the top 27 in order to remove any residue of oil 40 thereon. At this point in the process, the drops 40 have been absorbed into the absorbent 16, captured within the cage portion 30 of the housing 12.

Given the small pore size (comparatively, with respect to the non-porous plastic housing 12 and its vary large apertures 26, 32) and surface tension attraction, any oil content 40 is well contained. The diffuser system 10 may now be set on a shelf, dropped into a dresser drawer, put into a purse, be clipped to a ventilation vent in an automobile, or the like.

As a practical matter, the mouth 42 of the container 20 is usually located at the end of a neck 42 somewhat narrower than the container 20, itself. The neck 44 may be threaded, snap fit to the cap 18, or the like. In various embodiments, the mouth 42 may actually be obstructed by a fitting to render much easier the formation of droplets. For example, a narrow central spout may proceed from a fitting secured in the mouth 42. The spout may be surrounded by a channel that tends to retrieve any oil 40 that might be maintained on the spout by surface tension forces. In such an embodiment, a channel surrounding the spout will often have vents that drain back into the container 20 through the neck 44.

The housing 12 may be made operational on virtually any design for a cap 18, by being designed with lands 36 and grooves 38 appropriately fitted thereto. In certain embodiments, a housing 12 may even be manufactured in which the solid portion of the housing 12 below the cage 30 is, in fact, the cap 18. Connection to the neck 44 may be by threads, a snap-on detent, or the like. However, the illustrated embodiment is also a very useful configuration capable of retrofitting numerous standardized caps 18. Either configuration serves on a variety of standardized containers 20.

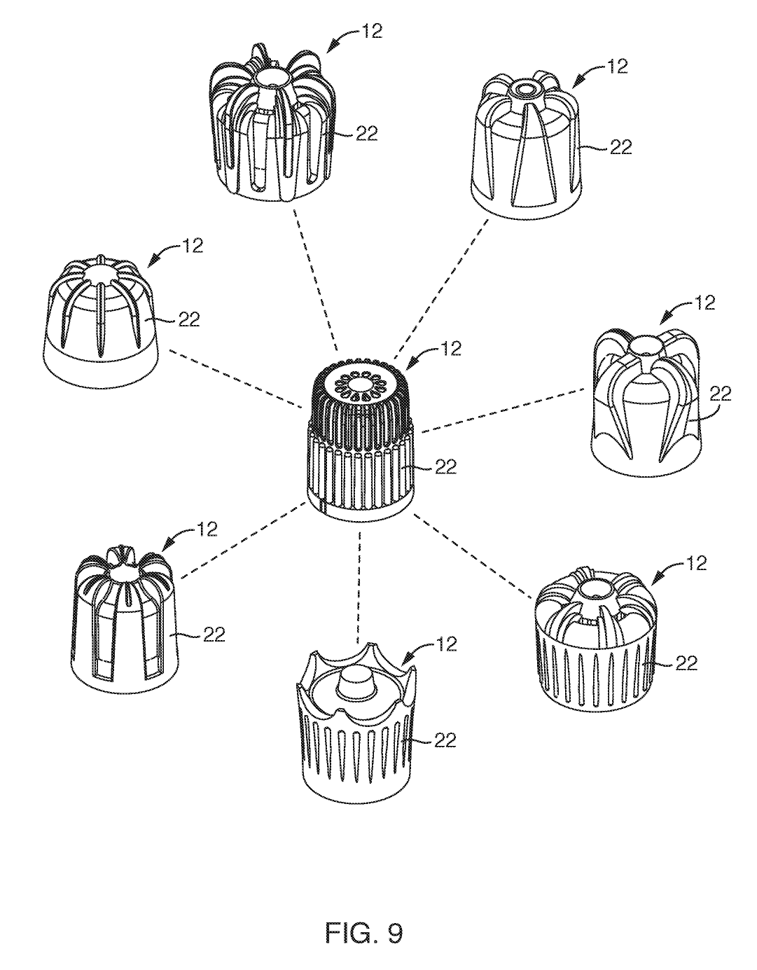

Referring to FIG. 9, while continuing to refer generally to FIGS. 1 through 13, in various embodiments, the housing 12 may take on any of a variety of shapes. Shapes may be useful for several purposes. For example, a shape may be suitable for operating as a trademark. Certain designers may create a shape, which shape becomes copyrighted, trademarked, or both to designate that designer as the origin of that housing 12.

Various alternative embodiments are illustrated in which the bars 28 and apertures 32 therebetween forming the cage 30 may be shaped in a decorative fashion to provide a more aesthetic, recognizable, or otherwise appealing shape. The housing 12 may be shaped as a sphere, a cylinder, crown, or the like.

Again, the relative area and shape of the apertures 32 or slots 32 between the bars 28 provide occlusion or closure containing the beads 16. They also effect the exposure of the beads 16 of absorbent 16 inside the cage 30 to ambient air. More exposure to air provides a corresponding, higher rate of evaporation or other volatilization of the oil 40 or other content 40 held by the absorbent 16.

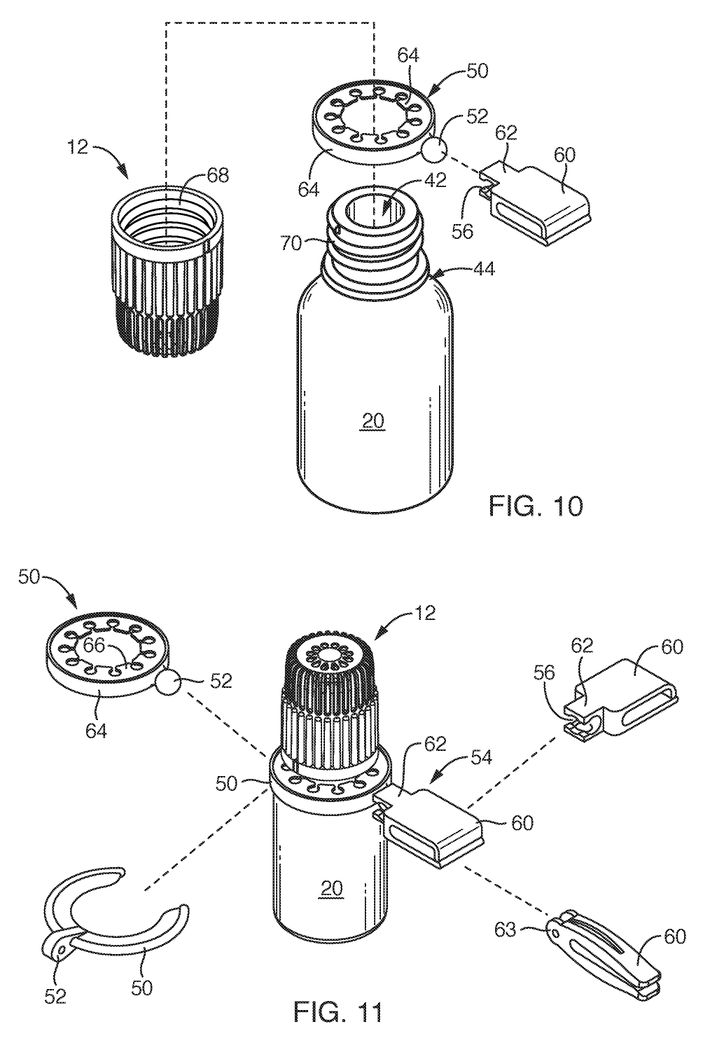

Referring to FIGS. 10 and 11, while continuing to refer generally to FIGS. 1 through 13, a system 10 in accordance with the invention may include a collar 50 formed to fit snuggly and remain stably located about the neck 44 of a container 20. In certain of the various illustrated embodiments, the collar may include an outer ring 64 and deflecting inner fingers 66 to snugly grasp the neck 44. The collar 50 may support include a trunnion 52, such as a ball 52 to be secured in a socket 56. A principal function of the collar 50 may be to secure a clip 60 to the bottle 20 or container 20.

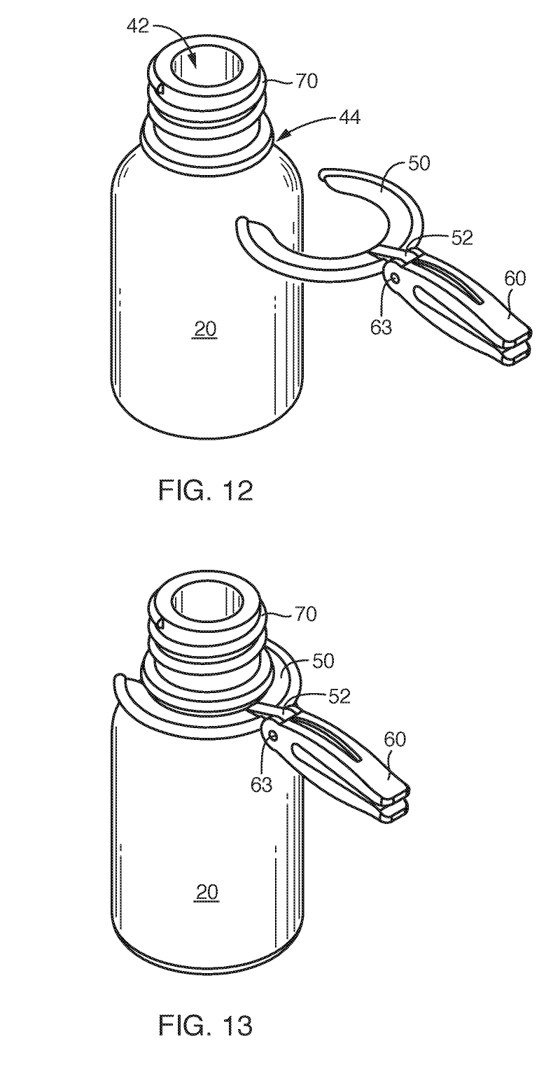

Referring to FIGS. 12 and 13, A series of clamps and fasteners, such as bolt, rivets, screws, or the like may replace the unitary socket 56. Likewise, pins, rods, and so forth may connect an alternative embodiment of a clip 60, by a clevis 63 to a suitable trunnion 52 configured as a stud 52 protruding from the collar 50.

Referring still to FIGS. 10-13, a collar 50 may be formed to have a ring 64 and fingers 66 of any suitable size and shape that will snap into place, passing over threads along the neck 44 of the container 20. Inwardly extending fingers 66 may secure the collar 50 between the change in diameter (shoulder), below the neck 44 of the container 20, and threads on the neck 44 of the container 20.

The trunnion 52 may be of any suitable shape, as illustrated. A spherical shape has certain benefits. For example, with a spherical or round ball 52 as a trunnion 52, a socket 56 may pivot with respect to the container 20 in three dimensions (degrees of freedom, axes of rotation). It may also rotate (spin, slide, rotate) about the neck 44 in order to align the clip 60 with an object for securement. In these ways, the system 10 may actually be clipped to a pocket within a purse or briefcase, clipped to a vent louver in an air conditioning system of a vehicle, secured to a curtain, object within a desk or dresser drawer, clothing or other object in a drawer, or the like.

A clip 60 may include a socket 56 formed in an extension 62 extending directly and rigidly from a clip 60. An interference fit between the trunnion 52 and the socket 56 of the extension 62 may benefit from sufficient flexibility in the stiff, if not quite rigid, extension 62 to firmly enclose the ball 52. This may permit securement and removal by a user. Accordingly, the clip 60 may be rotated about the ball 52 and tilted in order to permit the clip 60 to grip a suitable object and yet maintain the container 20 and the housing 12 in any suitable orientation.

Orientation is not necessarily a critical factor, inasmuch as the liquid is not free to exit the container 20 nor the housing 12 as drops. Rather, vapors may escape through the apertures 32 of the cage portion 30 of the housing 12, but only as vapor or individual molecules. In some embodiments, extremely small, even microscopic droplets might escape, but such operation is virtually impossible in quiescent, ambient air.

By the same token, a clevis 63 (See FIGS. 12 and 13) fitted and pinned to a stud-type trunnion 52 may only have one degree of freedom to pivot. Another degree of freedom is available by rotating the collar 50 with respect to the neck 44.

As discussed hereinabove, and as illustrated in FIGS. 10-11, the housing 12 may be formed to be the entire cap 18 and housing 12 as a single unit 10. This may be integrated by homogeneously molding threads 68 (see FIG. 10) into the housing 12. The entire housing may be homogeneously molded as a single piece. In such an embodiment, the seal 14 may be snapped or sealed (glued, melted, ultrasonically welded, etc.) in place after filling the cage 30 with absorbent 16. Alternatively, the system 10 may be assembled by molding a housing 12, filling the cage 30 with absorbent, then sealing the housing 12 to a pre-selected cap. The texturing on the outside surfaces of caps 18 tends to vary more (be less standardized) than the threads' 70 size and pitch. Thus, whether integrated by molding or by assembly, the housing 12 and cap 18 may contain the absorbent 16. Later closing of the housing 12 by a seal 14 provides the surface that will secure against the mouth 42 of the container 20 to close it.

The present invention may be embodied in other specific forms without departing from its purposes, functions, structures, or operational characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

What is claimed and desired to be secured by United States Letters Patent is:

1. A method for diffusing an aromatic substance, the method comprising:
providing a container;
providing a housing fitted as a cap to selectively seal and unseal the container, the housing having a cage open to ambient air and apertures effective to conduct a liquid into the cage;
providing an absorbent within the cage;
providing a liquid in the container having an aromatic constituents;
placing a quantity of the liquid from the container onto the apertures through an outer surface of the cage housing;
attracting by the absorbent through the apertures the quantity of the liquid;
sealing the container by placing the housing on the container; and
placing the container, capped with the housing in a location exposing the absorbent to ambient air passing through the cage.

2. The method of claim 1, further comprising selecting a polymeric material as the absorbent.

3. The method of claim 2, wherein the polymer is formed as granules.

4. The method of claim 3, wherein the granules are sized to be captured within the cage.

5. The method of claim 4, comprising providing slots through a sufficient portion of the cage to permit regular and continual passage of ambient air across the absorbent.

6. The method of claim 1, further comprising providing the housing in a size and shape configured to fit securely onto a separate cap portion.

7. The method of claim 1, further comprising forming the housing to be the cap in an integrated unit as a rigid body containing the absorbent.

8. The method of claim 1, further comprising removing residue of the liquid from an outer surface of the housing after placing the liquid on the apertures.

9. The method of claim 1, further comprising conditioning a closed space by placing the housing and container integrated together in a closed environment.

10. An apparatus comprising:
a container having a neck, a mouth opening opposite a bottom, and capable of holding a liquid selected to be aromatic;
a seal positioned to selectively open and close the mouth;
a housing operably connected to hold the seal proximate bottom end thereof and having a cage passing air therethrough proximate a top end thereof;
the housing operable to seal the cage away from the mouth in a closed configuration and to receive the liquid from the mouth into the cage in an open configuration; and
an absorbent captured within the cage and effective to receive a dose of the liquid therein directly from the mouth to expose the liquid to ambient air.

11. The apparatus of claim 10, wherein the cage is provided with slots exposing a significant fraction of a surface area of the absorbent to ambient air passing through the housing.

12. The apparatus of claim 10, wherein the absorbent is constituted as granules.

13. The apparatus of claim 12, wherein the absorbent is formed of a polymeric material.

14. The apparatus of claim 13, wherein the polymeric material is selected to secure the aromatic liquid interstitially within the polymeric material.

15. The apparatus of claim 14, wherein the polymeric material is formed in a rounded shape.

16. The apparatus of claim 15, wherein the granules are sized to render the housing effective to capture the granules within the cage.

17. An apparatus comprising:
a housing containing a wall perforated to form a cage capable of passing ambient air therethrough;
a floor separating the cage from a seal opposite;
an absorbent effective to retain the liquid within and evaporate it from the cage; and
a securement mechanism fitting the housing to a container containing the liquid in a manner effective to selectively seal the liquid in the container away from the cage and to open the container to apply a portion of the liquid therein to the absorbent.

18. The apparatus of claim 17, wherein the housing is further shaped to hold the seal against the container.

19. The apparatus of claim 17, wherein the housing forms a cap selectively securable to close the container.

20. The apparatus of claim 17, further comprising an engagement mechanism physically coupling the housing to the cap to move the cap in rigid body motion with the housing between a closed position threaded onto the container and an open position threaded off of the container.
